(19) United States
(12) Patent Application Publication
(10) Pub. No.: US 2019/0261886 A1
(43) Pub. Date: Aug. 29, 2019

(54) MEDICAL GUIDEWIRE WITH ELECTROMAGNETIC TRACKABLE ELEMENT

(71) Applicant: Lucent Medical Systems, Inc., Kirkland, WA (US)

(72) Inventors: Curtis S. King, Kirkland, WA (US); Samuel Peter Andreason, Kirkland, WA (US)

(21) Appl. No.: 16/288,012

(22) Filed: Feb. 27, 2019

Related U.S. Application Data
(60) Provisional application No. 62/636,093, filed on Feb. 27, 2018.

(51) Int. Cl. A61B 5/06; A61B 34/20; A61M 25/09

(52) U.S. Cl. CPC A61B 5/062; A61B 34/20; A61M 2025/09108; A61B 2034/2051; A61M 2025/09091; A61M 25/09

(57) ABSTRACT

A medical guidewire includes an electrically conductive core, a polymer tubing that encloses at least a portion of the electrically conductive core, and a trackable electromagnetic element formed in a distal end of the medical guidewire. The trackable electromagnetic element includes a coil. A first end of the coil is electrically coupled to an electrically conductive structure such as a metallic braid in the polymer tubing, and a second end of the coil is electrically coupled to the electrically conductive core.

### BACKGROUND

### Technical Field

The present disclosure is directed toward a medical guidewire. More particularly, but not exclusively, the present disclosure is directed toward a medical guidewire having a trackable electromagnetic coil integrated therein.

### Description of the Related Art

In many medical procedures, a medical practitioner accesses an internal cavity of a patient using a medical instrument. In some cases, the medical practitioner accesses the internal cavity for diagnostic purposes. In other cases, the practitioner accesses the cavity to provide treatment. In still other cases different therapy is provided.

Due to the sensitivity of internal tissues of a patient's body, incorrectly positioning the medical instrument within the body can cause great harm. Accordingly, it is beneficial to be able to precisely track the position of the medical instrument within the patient's body. However, accurately tracking the position of the medical instrument within the body can be quite difficult. The difficulties are amplified when the medical instrument is placed deep within the body of a large patient.

It is known that electromagnetic coil based medical instruments may be tracked as the instrument travels or remains stationary within the patient's body. For example, International Application No. PCT/US2017/014395 to Andreason et al. is entitled, LOW-FREQUENCY ELECTROMAGNETIC TRACKING. Here, systems, devices, and methods to track one or more low-frequency electromagnetic trackable structures are described. Embodiments of such methods include advancing a medical instrument into the body of a patient, wherein the medical instrument has at least one low-frequency electromagnetic apparatus affixed thereto. Each low-frequency electromagnetic apparatus includes at least one ferromagnetic core and at least one conductor, each of which may be dedicated or shared. The at least one conductor has a first portion arranged as a plurality of coils wound around a ferromagnetic core and a second portion arranged as a set of conductive leads. Embodiments of the method further include applying a low-frequency excitation signal to the set of conductive leads and detecting in real time, from outside the patient's body, at least one magnetic field produced by the low-frequency electromagnetic apparatus. In some embodiments, visual information is presented to track the motion or stationary position of the medical instrument inside the body of the patient based on the detected magnetic field. International Application No. PCT/US2017/014395 to Andreason et al. is incorporated herein by reference to the fullest extent allowed by law.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

Medical guidewires exist in various forms and are used in numerous applications. Some features in some medical guidewires include small size (diameter), flexibility, and in many cases, the ability to slip a device such as a medical catheter, stent, or the like over the guidewire, which allows a medical practitioner to position a device at a location in the body using the guidewire as a “pathway”, or “road map”. Because a device in some cases must slip over the guidewire, there are limitations for the electrical interface to an electromagnetic tracking element integrated with the guidewire.

A medical guidewire assembly may be summarized as including: an electrically conductive core; a polymer tubing enclosing at least a portion of the medical guidewire assembly; and a trackable electromagnetic element formed at a distal end of the electrically conductive core, wherein the trackable electromagnetic element includes a coil, and wherein a first end of the coil is electrically coupled to an electrically conductive structure enclosed by the polymer tubing and a second end of the coil is electrically coupled to the electrically conductive core.

At least a first electrical contact region may be formed at the first end of the coil, wherein the first electrical contact region is formed by a first plurality of coil windings that are shorting together; and at least a second electrical contact region may be formed at the second end of the coil, wherein the second electrical contact region is formed by a second plurality of coil windings that are shorting together. At least a third electrical contact region and a fourth electrical contact region may be formed at the proximal end of the medical guidewire assembly.

The electrically conductive core of the medical guidewire assembly may have a non-circular cross-section. The electrical coupling of the first end of the coil to the electrically conductive structure may be a soldered connection. The electrical coupling of the first end of the coil to the electrically conductive structure may be a laser weld connection. The electrically conductive structure and the polymer tubing may be integrated into a single structure. The electrically conductive structure may be a metallic braid. The medical guidewire may have a diameter of less than three millimeters, and the medical guidewire may have a length between about 0.5 millimeters and about 3000 millimeters.

A method to make a medical guidewire assembly bearing a trackable electromagnetic element may be summarized as including: providing an electrically conductive core, the electrically conductive core having a first outer insulating layer; providing an electrically conductive guidewire structure; forming a coil at a distal end of the electrically conductive core, the coil formed from a wire having a second outer insulating layer; electrically coupling a first end of the coil to the electrically conductive guidewire structure; and electrically coupling a second end of the coil to the electrically conductive core.

The method may include enclosing at least a portion of the electrically conductive guidewire with a polymer tubing layer. Electrically coupling the first end of the coil to the electrically conductive guidewire structure may include: ablating a portion of the second outer insulating layer of the wire at the first end of the coil to expose an electrically conductive portion of the wire; exposing an electrically conductive portion of the electrically conductive guidewire; and forming an electrically conductive joint between the exposed electrically conductive portion of the wire and the exposed electrically conductive portion of the electrically conductive guidewire. Electrically coupling the second end of the coil to the electrically conductive core may include: ablating a portion of the second outer insulating layer of the wire at the second end of the coil to expose an electrically conductive portion of the wire; ablating a portion of the first outer insulating layer of the electrically conductive core to expose an electrically conductive portion of the electrically conductive core; and forming an electrically conductive joint between the exposed electrically conductive portion of the wire and the exposed electrically conductive portion of the electrically conductive core. The electrically conductive guidewire structure may be a metallic braid.

A medical guidewire method may be summarized as including: identifying a location of interest inside a body of a patient; introducing a guidewire into the body of the patient, the guidewire having an electrically conductive core, and a trackable electromagnetic element formed in a distal end of the medical guidewire; advancing the distal end of the medical guidewire into the body of the patient; magnetically tracking the distal end of the medical guidewire advancing in the body of the patient; and presenting real time visual imagery representing a path of progress of the distal end of the medical guidewire advancing in the body of the patient.

The method may include stopping the advancing when, based on the real time visual imagery, the distal end of the medical guidewire has reached the location of interest inside the body of the patient. Advancing the distal end of the medical guidewire into the body of the patient may include stopping the advancing; reversing the direction of progress of the distal end of the medical guidewire; and restarting the advancing of the distal end of the medical guidewire, and the stopping, reversing, and restarting may be visually presented in the real time visual imagery. Advancing the distal end of the medical guidewire into the body of the patient may include advancing the distal end of the medical guidewire through a fluid-carrying conduit before reaching the location of interest inside the body of the patient. The fluid-carrying conduit may be a blood-carrying conduit.

This Brief Summary has been provided to introduce certain concepts in a simplified form that are further described in detail below in the Detailed Description Except where otherwise expressly stated, the Brief Summary does not identify key or essential features of the claimed subject matter, nor is it intended to limit the scope of the claimed subject matter.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is an embodiment of a first medical guidewire assembly 10 having a trackable electromagnetic element integrated therein;

FIG. 2 is the embodiment of a first medical guidewire assembly 10 having particular detailed portions identified;

FIG. 3 is the embodiment of a first medical guidewire assembly 10 in more detail;

FIG. 4 is detail “A” in the trackable electromagnetic element embodiment 32A of FIG. 2;

FIG. 5 is a cross sectional view across detail “F” of the trackable electromagnetic element embodiment 32A of FIG. 4;

FIG. 6 is the embodiment of a first medical guidewire assembly 10 in more detail;

FIG. 7 is a cross sectional view across detail “G” of the medical guidewire assembly 10 embodiment of FIG.;

FIG. 8 is detail “H” in the medical guidewire assembly 10 embodiment of FIG. 6;

FIG. 9 is detail “I” in the medical guidewire assembly 10 embodiment of FIG. 6;

FIG. 10 is detail “J” in the medical guidewire assembly 10 embodiment of FIG. 6;

FIG. 11 is detail “B” in the trackable electromagnetic element embodiment 32A of FIG. 2;

FIG. 12 is a cross sectional view across detail “K” of the trackable electromagnetic element embodiment 80 of FIG. 11;

FIG. 13 is a front view of a metallic braid reinforced polymer tubing;

FIG. 14 is a top view of the metallic braid reinforced polymer tubing with ablation sections;

FIG. 15 is detail “L” in the metallic braid reinforced polymer tubing embodiment of FIG. 14 showing the ablation region in more detail;

FIG. 16 is a braid reinforced polymer tubing embodiment installed over a medical guidewire assembly having a trackable electromagnetic element assembly;

FIG. 17 is a cross sectional view across detail “M” of the medical guidewire assembly embodiment of FIG. 16;

FIG. 18 is detail “N” in the medical guidewire assembly embodiment of FIG. 17;

FIG. 19 is an exposed section of the metallic braid reinforced polymer tubing embodiment over a trackable electromagnetic element embodiment and with a solder joint;

FIG. 20 is detail “O” in the exposed section of the metallic braid reinforced polymer tubing embodiment of FIG. 19;

FIG. 21 is detail “C” in the trackable electromagnetic element embodiment 32A of FIG. 2;

FIG. 22 is a cross sectional view across detail “P” of the proximal end of a guidewire with a solder joint electrical contact embodiment of FIG. 21;

FIG. 23 is detail “Q” in the proximal end of a guidewire with a solder joint electrical contact embodiment of FIG. 22;

FIG. 24 is detail “D” in the trackable electromagnetic element embodiment 32A of FIG. 2;

FIG. 25 is a cross sectional view across detail “R” of the proximal end of a guidewire embodiment of FIG. 24;

FIG. 26 is detail “E” in the trackable electromagnetic element embodiment 32A of FIG. 2;

FIG. 27 is a cross sectional view across detail “S” of the proximal end of a guidewire embodiment of FIG. 26;

FIG. 28 is a guidewire assembly 10A embodiment used as a core for a medical guidewire;

FIG. 29 is a medical guidewire assembly 10B embodiment with a coil pipe overtube;

FIG. 30 is a guidewire assembly 10C embodiment having a metallic braid woven over an entire length of a guide wire;

FIG. 31 is detail “T” in the guidewire assembly embodiment 10C of FIG. 30;

FIG. 32 is detail “U” in the guidewire assembly embodiment 10C of FIG. 30;

FIG. 33 is a guidewire assembly final product embodiment;

FIGS. 34A-34F are cross sectional views of core wire embodiments;

FIG. 35 is another guidewire assembly embodiment;

FIG. 36A is a low-frequency electromagnetic tracking system; and

FIG. 36B illustrates a medical environment including a low-frequency electromagnetic tracking system for detecting the position of a guidewire assembly with a trackable electromagnetic element.

### DETAILED DESCRIPTION

Guidewires are used extensively in numerous medical procedures. A guidewire is used to facilitate the delivery of a device or devices into one or more specific locations within the body of a patient (e.g., human or other animal), for therapeutic, corrective, diagnostic, or other such procedures. For example, in a catheter placement procedure, a medical practitioner may desire to position a catheter in a particular site in the patient's body. In at least some cases, the location of interest includes a blood vessel, vein, artery, or some other blood-carrying conduit. In these or other cases, the guidewire is passed through a blood-carrying conduit or a conduit that carries another bodily fluid toward a destination location of interest such as the heart, brain, spinal cord, lungs, stomach, kidney, or some other location in the patient's body. In still other cases, the guidewire is passed through a portion of the patient's body via some other path.

To perform the procedure, the medical practitioner may first introduce a guidewire to the vessel and follow the path of the guidewire under ultrasound or fluoroscopic vision. The medical practitioner manipulates the guidewire, advancing its position in the vessel until the site of interest is reached. At this point, the medical practitioner will pass the catheter over the guidewire, using the wire as a “path” to position the body and distal tip of the catheter in the desired location.

Known guidewires exist in a range of sizes (e.g., diameters of 0.014 inch, 0.018 inch, 0.035 inch, 0.08 inch, 0.125 inch, and other diameters). It will be recognized, however, that the present disclosure is not limited to a guidewire of any particular size, braiding, stiffness, or any other such characteristic. Known guidewires are constructed using various materials. The guidewires of the present disclosure include an electromagnetic element, but such guidewires are not otherwise limited to any particular material and conventional guidewire materials may be included in the embodiments described herein.

It is common for many guidewires to have coatings to reduce friction during insertion. The guidewires of the present disclosure may include these coatings, but the present guidewires do not require the coatings. Also, many wire designs incorporate mechanical design elements that allow some degree of mechanical manipulation (steering, torquing, etc) by the medical practitioner during installation. The guidewires of the present disclosure may include these mechanical design elements, but the present guidewires do not require these mechanical design elements. In at least some cases, where the mechanical design elements are includes, the guidewires of the present disclosure arrange such design elements as electromechanical design elements. That is, the design element (e.g., a wire braid, an interwoven wire, or some other like element) may be electrically coupled to at least one portion of an electromagnetic element in the distal end of the guidewire, and the design element may be used to both mechanically manipulate the guide wire and electrically stimulate the electromagnetic element.

In practice, conventional guidewires are frequently placed under direct fluoroscopic (e.g., x-ray) visualization. This is problematic for medical practitioners, and to some degree patients, because of the potential for radiation over-exposure. To address this concern, guidewires of the present disclosure incorporate a small receiver coil, or some other electronic element at their tip that enable electromagnetic or electronic detection/navigation functionality. One challenge with these types of devices that has been overcome by the inventors is the ability to make the electrical connection to a small sized device, while preserving the ability to slip another device (e.g., a catheter, a drainage tube, a feeding tube, a camera device, a therapy delivery device, or some other device) over the guidewire. The present disclosure describes embodiments of an electromagnetic tracking element integrated into medical guidewire devices of all types. Embodiments represent potentially more durable, manufacturable approaches to construction of navigable guidewires that bear an integrated, trackable electromagnetic element.

Medical guidewires are, generally speaking, a thin, flexible wire structure passed into the body of a patient; often in a confined or complex or otherwise tortuous space. Once placed, the guidewire is used to guide, escort, or otherwise facilitate placement of one or more selected medical devices. The guidewire can be used, for example, to position an intravenous catheter, an endotracheal tube, a central venous line, a gastric feeding tube, or the like. The guidewire may also be used to guide or deliver one or more therapeutic agents (e.g., a cautery instrument, a biopsy forceps tool, or some other agent) to a localized site such as a tumor.

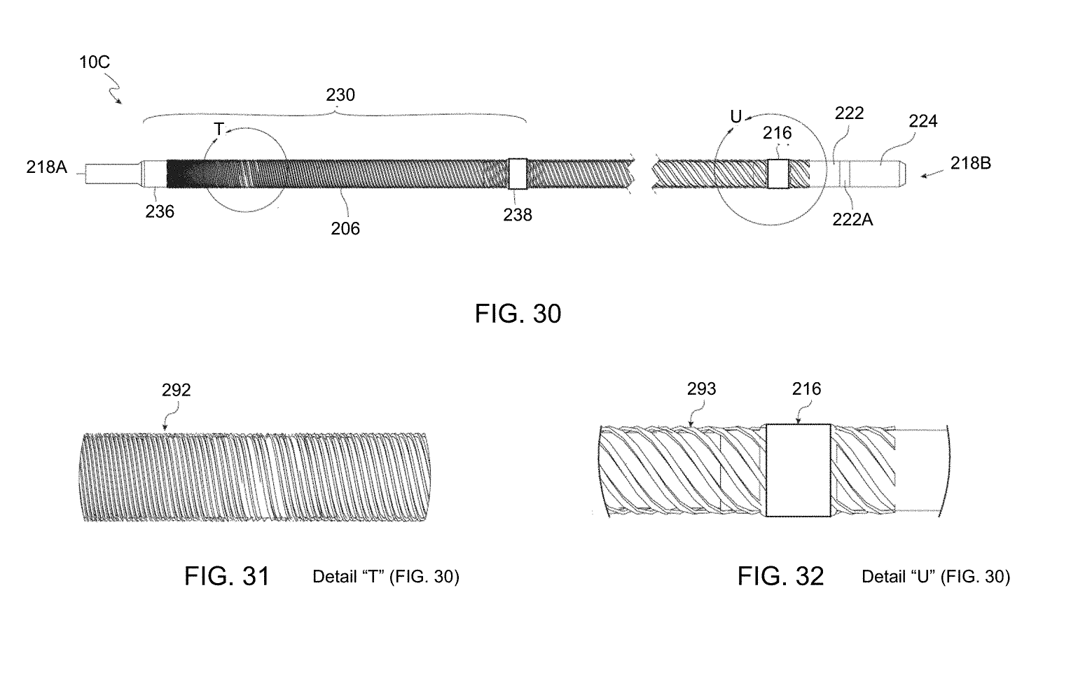

Medical guidewires may have a diameter of about 0.2 millimeters (mm) to about 3.0 mm (FIG. 30F). Other smaller and larger diameters are contemplated. Medical guidewires may have any length. At least some of the guidewires contemplated herein have a length of between about 0.5 mm to about 3000 mm (FIG. 30F). Other shorter and longer lengths are contemplated. Medical guidewires may have cylindrical shapes, conical shapes, helical shapes, or some other shape. Stated differently, the medical guidewires described herein may have a cross-section that is circular, ovular, square, rectangular, triangular, hexagonal, octagonal, polygonal, or having any other suitable cross-section (e.g., FIGS. 30A-30F). Coils in a medical wire, if the medical wire is so-constructed, may have any pitch and any distance between individual coils including variable pitch and variably spaced coils. Medical wires may be soft or stiff by any definition of such terms and by any requirement of a medical practitioner, and along these lines, medical wires may be as springy, elastic, torque-resistant, biased or have any other shape-related characteristics as a medical practitioner desires. Some medical guidewires are wholly or in part straight, pre-shaped, angled, or otherwise formed. Some medical guidewires can be steered with integrated or added steering structures.

In the present disclosure a medical guidewire has an electromagnetic trackable element integrated therein. Embodiments of such medical guidewires are discussed in the present disclosure along with methods of making the medical guidewires.

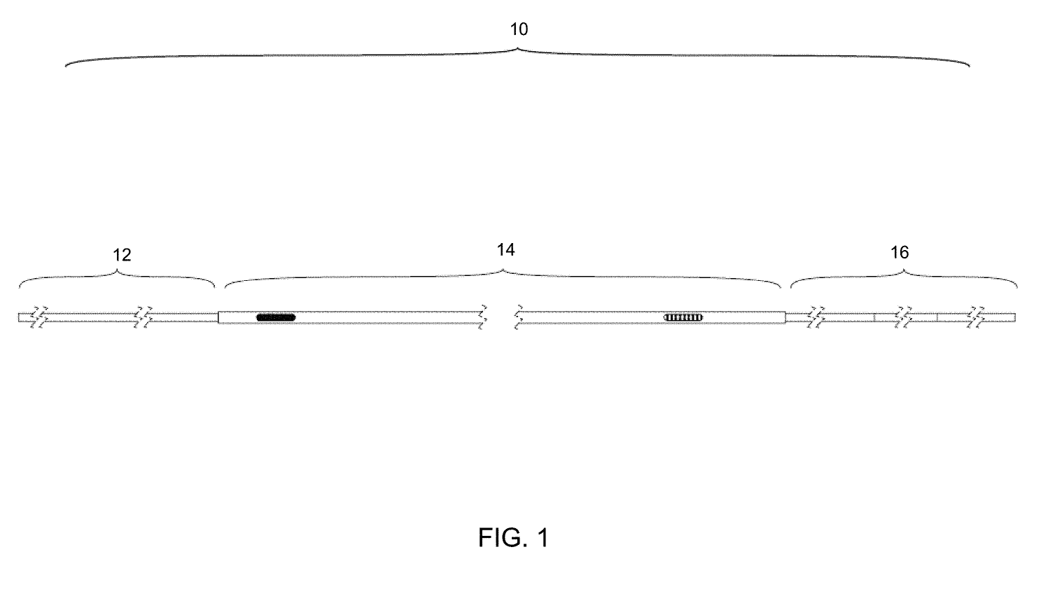

FIG. 1 is an embodiment of a first medical guidewire assembly 10 having a trackable electromagnetic element integrated therein. The assembly includes an electromagnetic tracking element 12 at its distal tip, an electrically conductive (e.g., metallic) braided tubing (overtube) 14, and an electrical contacts section 16 for the trackable electromagnetic element.

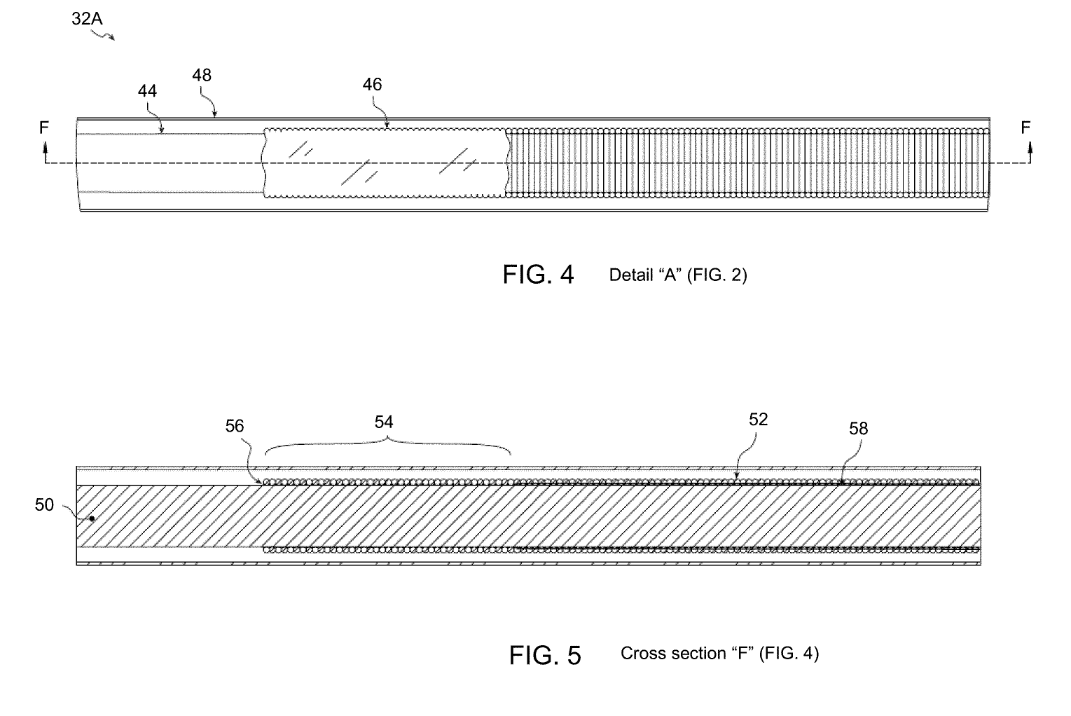

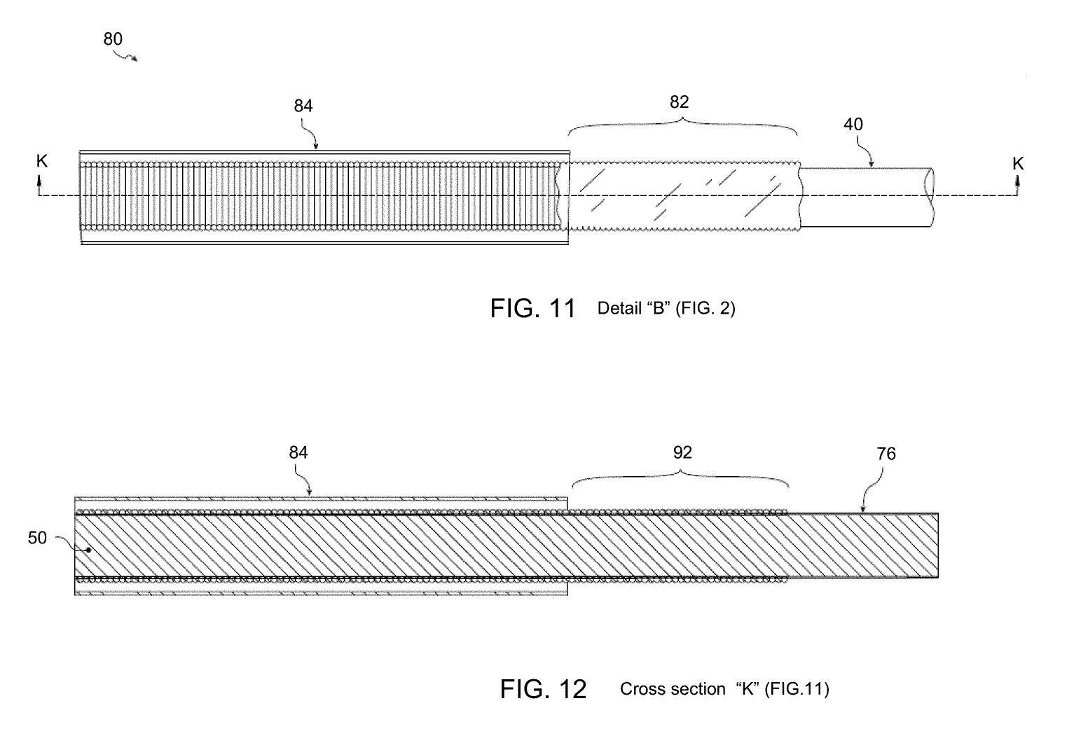

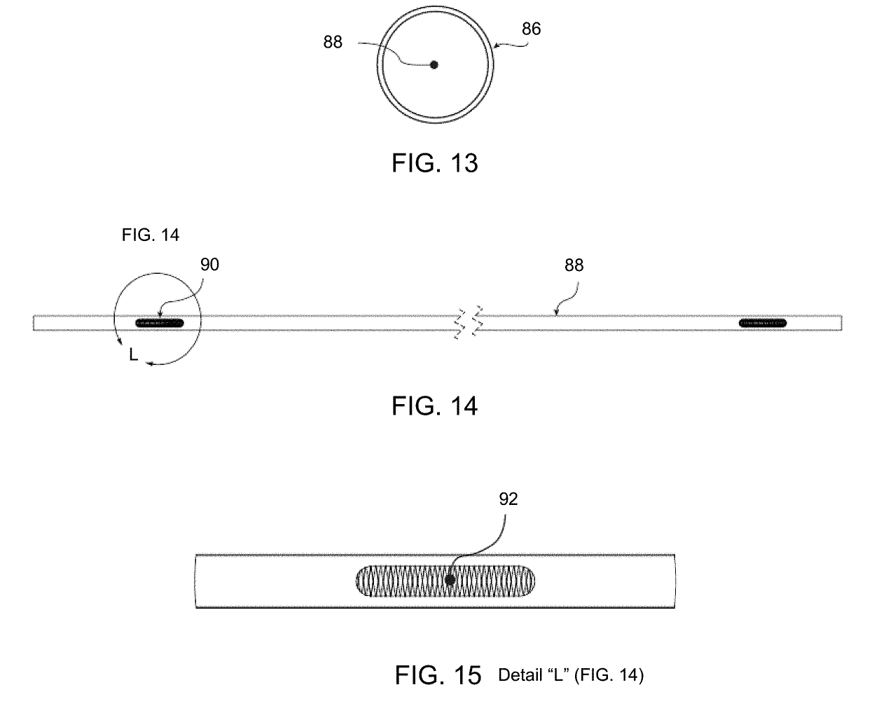

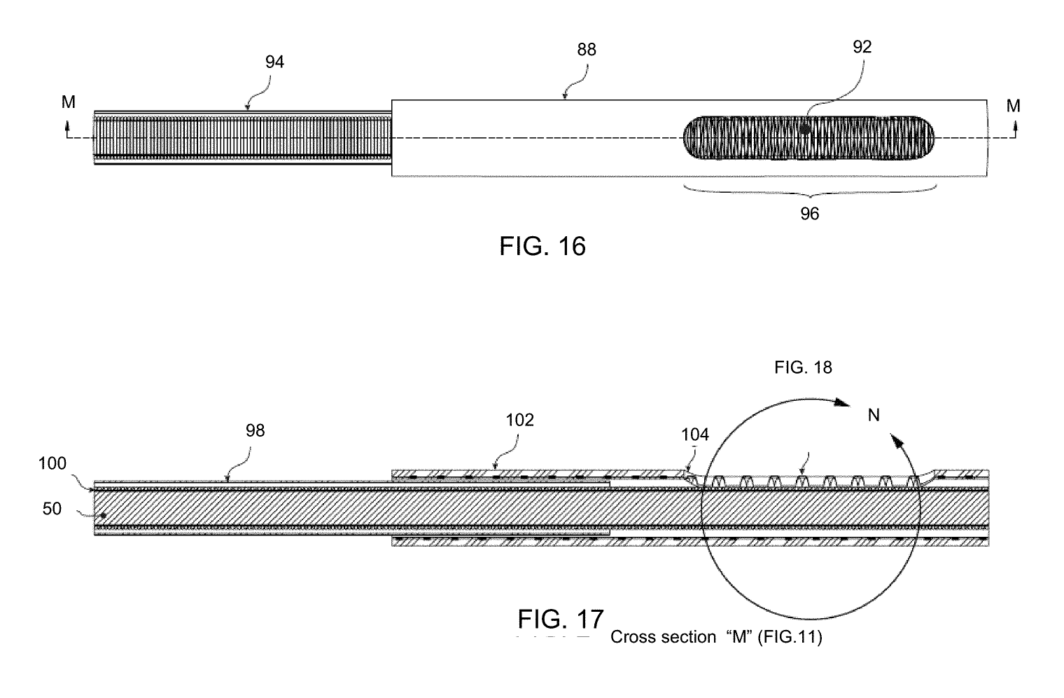

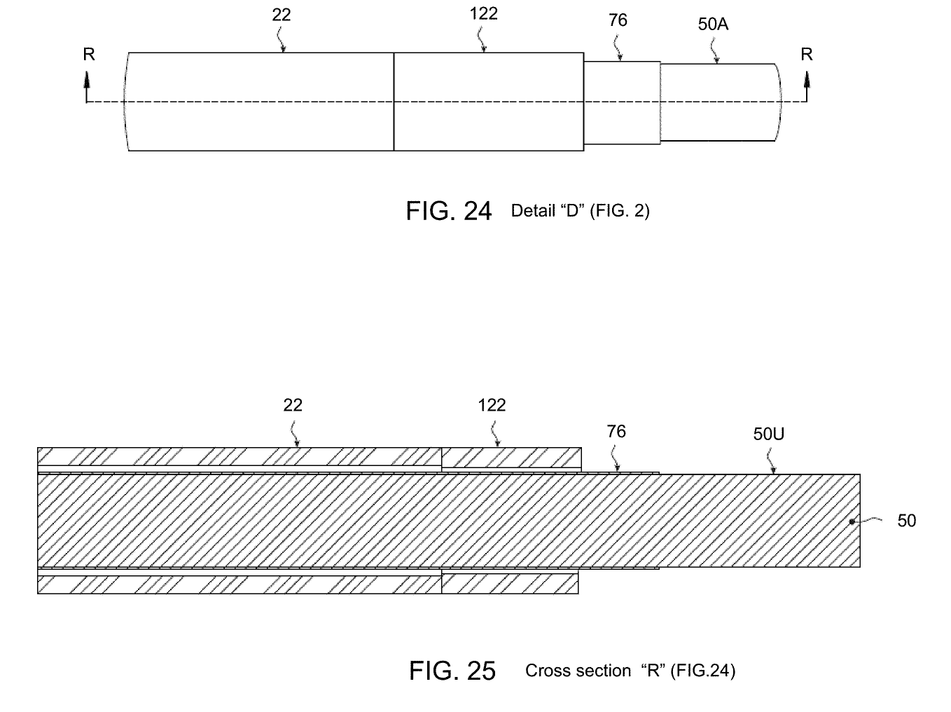

FIG. 2 is the embodiment of a first medical guidewire assembly 10 having particular detailed portions identified. A trackable electromagnetic element at the distal tip 18 is illustrated in detail “A” (FIG. 4). An electrically conductive (e.g., metallic) braided tubing section 20 having “exposed windows” is illustrated in detail “B” (FIG. 11″ and detail “C” (FIG. 21). A first electrical contact 22 for the trackable electromagnetic element is shown. An electrically insulating contact spacer is shown in detail “D” (FIG. 24). A second electrical contact 26 for the trackable electromagnetic element is shown in detail “E” (FIG. 26).

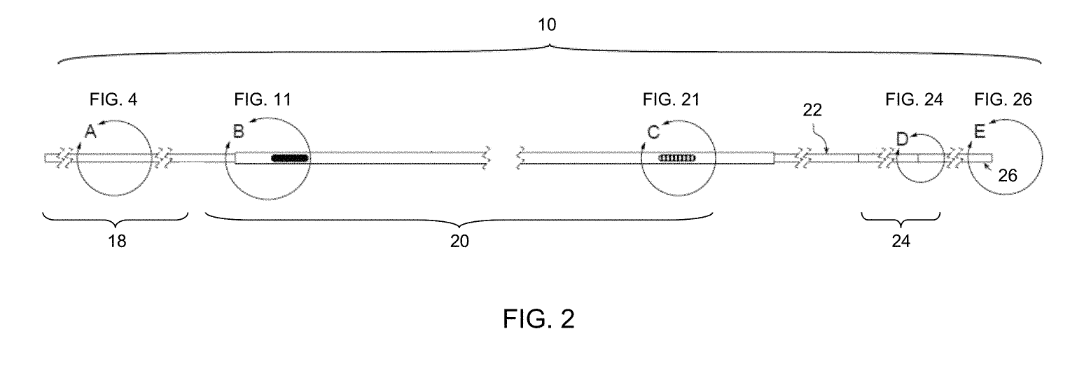

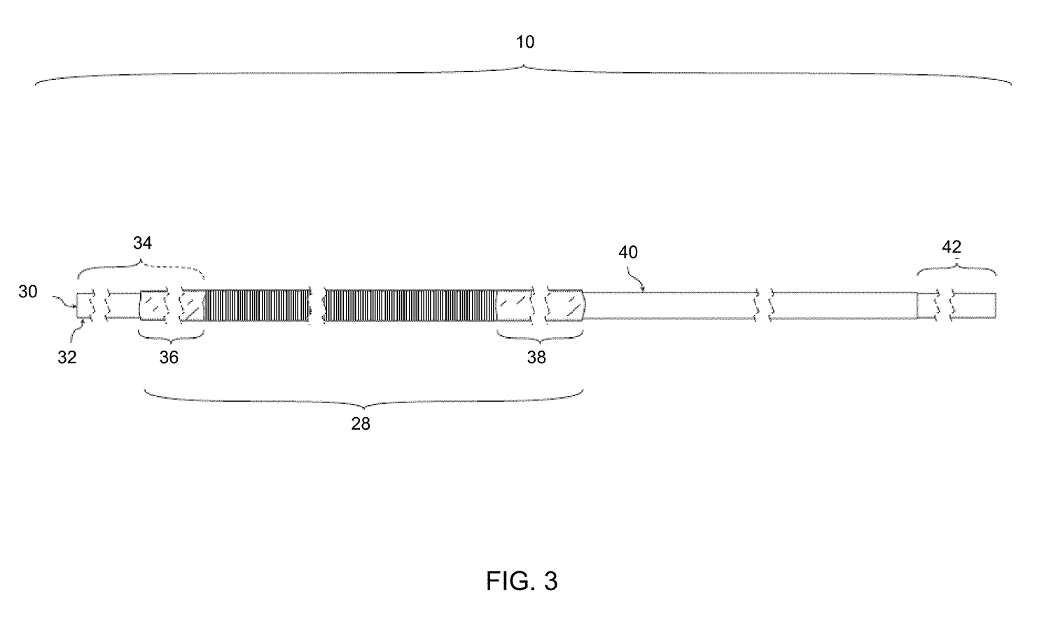

FIG. 3 is the embodiment of a first medical guidewire assembly 10 in more detail. The trackable electromagnetic element is represented as a coil wound on a core with proximal and distal solder sections 28. The trackable electromagnetic element has a core wire 30. The core wire 30 is electrically conductive. The core wire 30 includes an outer insulating layer over at least a portion of its length. The distal tip has a non-insulated core wire section 32. In some cases, insulation that is present when the core wire 30 is manufactured is removed from a first section 34 exposing a distal end of the core wire extending some distance under the coil. In other cases, core wire 30 may be manufactured without insulation at a distal first section 34. A distal solder section 36 of the trackable electromagnetic element is shown, and a proximal solder section 38 of the trackable electromagnetic element is shown. The structure includes an insulated core wire 40. Insulation is removed from a second section 42 exposing a proximal end of the core wire. In at least some cases, removing the insulation as described herein includes ablating the insulation. The insulation may be ablated by heating (e.g., heating during soldering, heating during laser welding, or heating in another way), by abrasion (e.g., scraping, rubbing, stripping, or the like), by crimping, or by some other technique.

FIG. 4 is detail “A” in the trackable electromagnetic element embodiment 32A of FIG. 2. A trackable electromagnetic element 32A having a tubing sheath is shown in the distal tip of the first medical guidewire assembly 10. A portion where insulation is removed 44 (e.g., by ablated) at the distal end of core wire and extending some distance under the trackable electromagnetic element is shown. A solder section 46 is shown at the distal end of the trackable electromagnetic element. A tubing sheath 48 covers the trackable electromagnetic element.

FIG. 5 is a cross sectional view across detail “F” of the trackable electromagnetic element embodiment 32A of FIG. 4. A core wire 50 of the trackable electromagnetic element and a coil 52 of the trackable electromagnetic element are depicted. In a distal solder section of the trackable electromagnetic element, a plurality of coil windings are electrically connected (i.e., shorted via soldering, laser welding, or some other suitable technique) to one another 54. In a non-insulated core section at the distal end of the trackable electromagnetic element, a solder section 56 of the coil is electrically connected (i.e., shorted) to the core 50. Electrically isolated (e.g., insulated by an outer layer of core wire insulation) coil windings 58 are shown. These coil windings are electrically isolated from one another and from the core via an outer insulating layer on the wire used to form the coil windings 58.

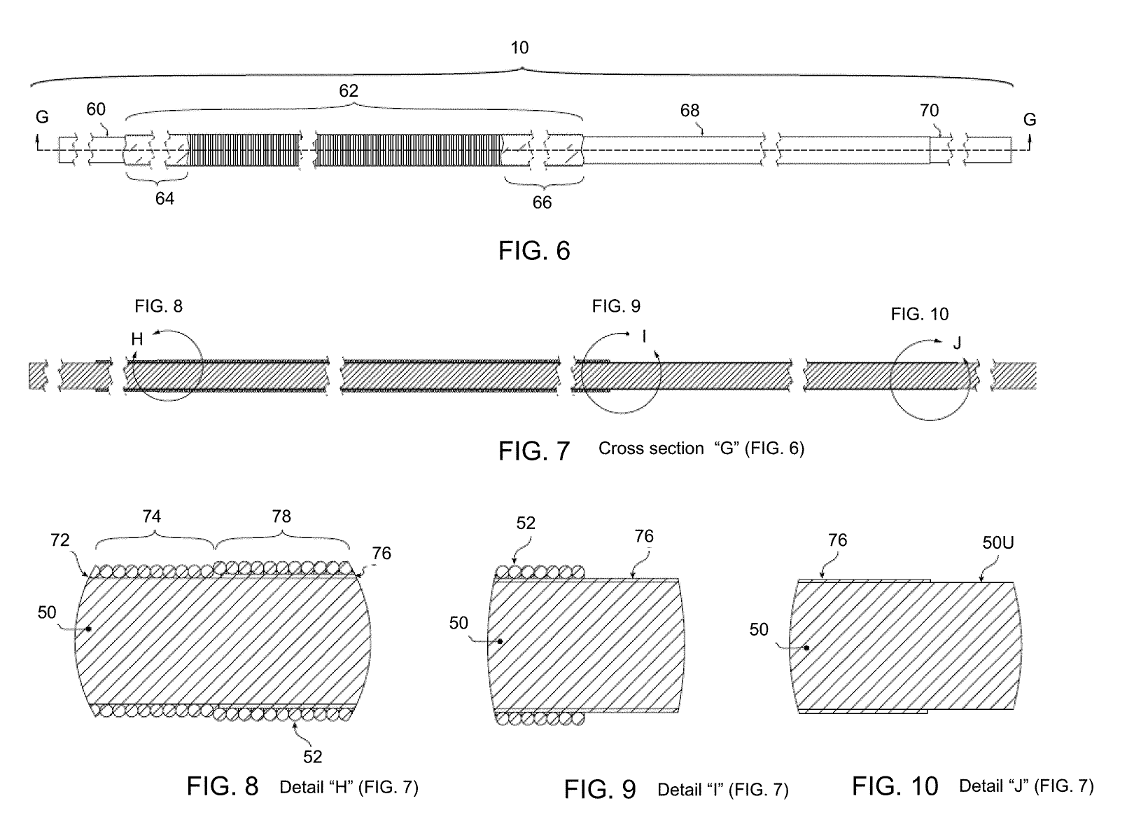

FIG. 6 is the embodiment of a first medical guidewire assembly 10 in more detail. A non-insulated distal tip 60 of a core wire is depicted as is a trackable electromagnetic element assembly with distal and proximal solder sections 62. A distal solder section 64 of the trackable electromagnetic element is shown where the distal end of the element is shorted to the core (e.g., soldered, laser welded, or electrically connected in another way). In a proximal solder section 66, the coil is isolated from core. An insulated section 68 of the core wire is illustrated and a non-insulated, proximal end 70 of the core wire is illustrated.

FIG. 7 is a cross sectional view across detail “G” of the medical guidewire assembly 10 embodiment of FIG. 6. Detailed areas “H,” “I,” and “J” are identified in FIG. 7.

FIG. 8 is detail “H” (FIG. 7) in the medical guidewire assembly 10 embodiment of FIG. 6. FIG. 8 shows a distal solder section of a trackable electromagnetic element embodiment. The trackable electromagnetic element includes a core wire 50, a coil 52, a non-insulated core wire 72, a coil section that is electrically shorted to the core 74, an insulation layer 76 of the core wire, and a coil section that is electrically isolated from the core 78.

FIG. 9 is detail “I” (FIG. 7) in the medical guidewire assembly 10 embodiment of FIG. 6. FIG. 9 shows a proximal solder section of the coil of the trackable electromagnetic element embodiment. In the proximal solder section, a core wire 50 of the trackable electromagnetic element embodiment is shown. A proximal solder section 52 of the trackable electromagnetic element embodiment is electrically isolated from core 50. The insulation layer 76 of the trackable electromagnetic element core wire 50 is also shown.

FIG. 10 is detail “J” (FIG. 7) in the medical guidewire assembly 10 embodiment of FIG. 6. FIG. 10 shows a proximal end of the core wire of the trackable electromagnetic element embodiment. In FIG. 10, a core wire 50, an insulation layer 76 of the core wire, and a non-insulated section 50U of the core wire are illustrated.

FIG. 11 is detail “B” in the trackable electromagnetic element embodiment 32A of FIG. 2. The figure shows a proximal section of the coil of a trackable electromagnetic element embodiment. In FIG. 11, a core wire assembly 80 of the trackable electromagnetic element is shown along with an insulated section of core wire 40, a proximal solder section 82 of the coil, and a tubing sheath 84 covering the coil.

FIG. 12 is a cross sectional view across detail “K” of the trackable electromagnetic element embodiment 80 of FIG. 11. A proximal section of the trackable electromagnetic element coils is depicted. The figure shows core wire 50, an outer insulation layer 76 of the core wire, a proximal solder section 92 of the coil of the trackable electromagnetic element, and a tubing sheath 84 covering the coil. The tubing sheath 84 may be a nylon or some other polymer, a rubber-based material, a silicon-based material, or any other suitable material. For brevity, the tubing sheath 84 is referred to as a polymer tubing, but one of skill in the art will recognize that such tubing in some embodiments may include a tubing of another suitable material.

FIG. 13 is a front view of an electrically conducive material, such as a metallic braid, reinforced polymer tubing. The tubing includes a wall 86 of the metallic braid reinforced polymer tubing and a center lumen 88. In some cases, of the reinforced polymer tubing, the electrically conductive material and the tubing material are integrated. In some other cases, the electrically conductive material and the tubing material are separate and distinct. For example, the electrically conductive material may be arranged over the core wire at a first time, and the tubing material may be arranged over the combination of the electrically conductive material and core wire at a second time.

FIG. 14 is a top view of the electrically conductive material (e.g., metallic braid) reinforced polymer tubing with ablation sections. The electrically conductive material (e.g., metallic braid) reinforced polymer tubing 88 is expressly identified along with an ablation window 90 (e.g., an exposed section) in the polymer tubing wall at the distal end of the trackable electromagnetic element is shown. In at least some cases, the ablation window is formed by removing an outer layer of insulation by heating (e.g., heating during soldering, heating during laser welding, or heating in another way), by abrasion (e.g., scraping, rubbing, stripping, or the like), by cutting, or by some other technique.

FIG. 15 is detail “L” in the electrically conductive material (e.g., metallic braid) reinforced polymer tubing embodiment of FIG. 14 showing the ablation region in more detail. The ablated section 92 of the polymer tubing exposes the electrically conductive material (e.g., metallic braid) of the tubing or otherwise below the tubing.

FIG. 16 is a braid reinforced polymer tubing embodiment installed over a medical guidewire assembly having a trackable electromagnetic element assembly. The assembly includes a length of metallic braid reinforced polymer tubing 88 and an ablated section 92 of the polymer tubing, which exposes the metallic braid. A proximal end 94 of the trackable electromagnetic element assembly is shown. A proximal end solder section 96 includes an ablated section of the metallic braid reinforced polymer tubing, which exposes a solder section of the trackable electromagnetic element coil. The solder section may interchangeably be referred to herein as an electrical contact section. Electrical contact in the solder section may be performed by soldering, laser welding, or any other suitable process to form the electrical connection.

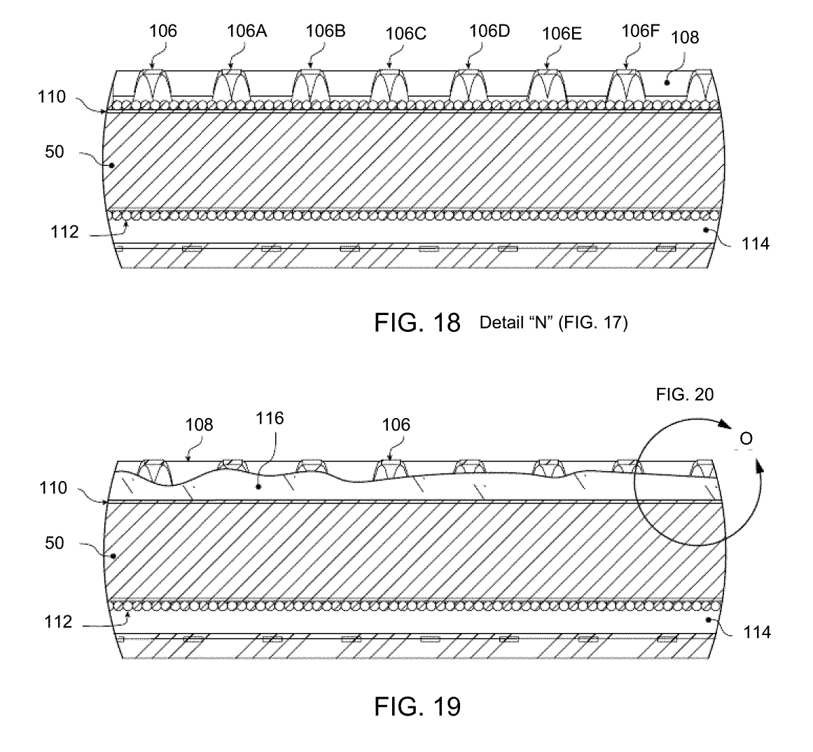

FIG. 17 is a cross sectional view across detail “M” of the medical guidewire assembly embodiment of FIG. 16. The figure shows a proximal end of the trackable electromagnetic element coil formed in the braid reinforced polymer tubing. Specifically, a tubing sheath 98 covering the coil is shown. Windings of the trackable electromagnetic element coil 100 wound over the insulated core wire and the core wire 50 itself are shown. The outer layer of the metallic braid reinforced polymer tubing 102 is distinguished from the ablated section 104 of the braid reinforced polymer tubing. The arrangement is shown along with the exposed electrically conductive structure (e.g., metallic braid) 106.

FIG. 18 is detail “N” in the medical guidewire assembly embodiment of FIG. 17. FIG. 18 shows an exposed section of the braid reinforced polymer tubing that is arranged over the coil and core wire assembly of the trackable electromagnetic element embodiment. The assembly includes a polymer wall 108 of the braid reinforced polymer tubing, the core wire 50, core wire insulation 110, and the coil 112 of the trackable electromagnetic element. Portions 106A-106F of the exposed metallic braid 106 are shown along with a center lumen 114 of the metallic braid reinforced polymer tubing. In at least some cases, it is recognized that the electrically conductive structure (e.g., metallic braid) and the tubing material are integrated. In some other cases, the electrically conductive structure (e.g., metallic braid) and the tubing material are separate and distinct. For example, the electrically conductive material may be arranged over the core wire at a first time, and the tubing material may be arranged over the combination of the electrically conductive material and core wire at a second time.

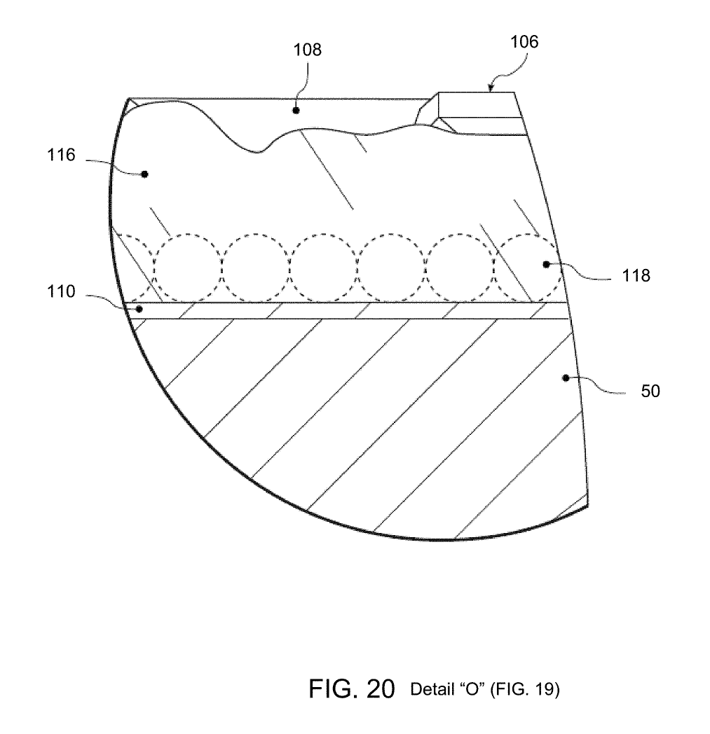

FIG. 19 is an exposed section of the metallic braid reinforced polymer tubing embodiment over a trackable electromagnetic element embodiment and with an electrical coupling (e.g., solder joint, laser weld, or some other electrical connection). In the section, the polymer wall 108 of the braid reinforced polymer tubing and an exposed metallic braid 106 are shown. The core wire 50, core wire insulation 110, and the coil 112 of the trackable electromagnetic element embodiment are illustrated. A detailed view of a solder joint 116 creates an electrical connection between the proximal end of the trackable electromagnetic element coil and the braid within the polymer tubing. The solder joint 116 is formed through the ablated section of the tubing. The center lumen 114 of the metallic braid reinforced polymer tubing is shown.

FIG. 20 is detail “O” in the exposed section of the metallic braid reinforced polymer tubing embodiment of FIG. 19. FIG. 20 shows the solder joint at the proximal end of the trackable electromagnetic element coil in electrical contact with the metallic braid. For reference, the core wire 50, core wire insulation layer 110, and wall 108 of the braid reinforced polymer tubing are shown. A single exposed metallic braid 106 is shown in electrical contact 118 with the coil of the trackable electromagnetic element through a solder joint 116.

FIG. 21 is detail “C” in the trackable electromagnetic element embodiment 32A of FIG. 2. The figure shows the proximal end of a guidewire with a solder joint electrical contact embodiment. In other embodiments, the electrical contact will be formed by a laser weld or other electrical contact means. The embodiment of FIG. 21 includes metallic braid reinforced polymer tubing 102, an ablated window 92 exposing the metallic braid, and the exposed metallic braid 106. A first electrical contact region 22A includes a conductive (e.g., metallic) tube-like structure installed within the lumen of braid reinforced polymer tubing. An electrical connection with metallic braid is shown. The insulated core wire 50 is identified.

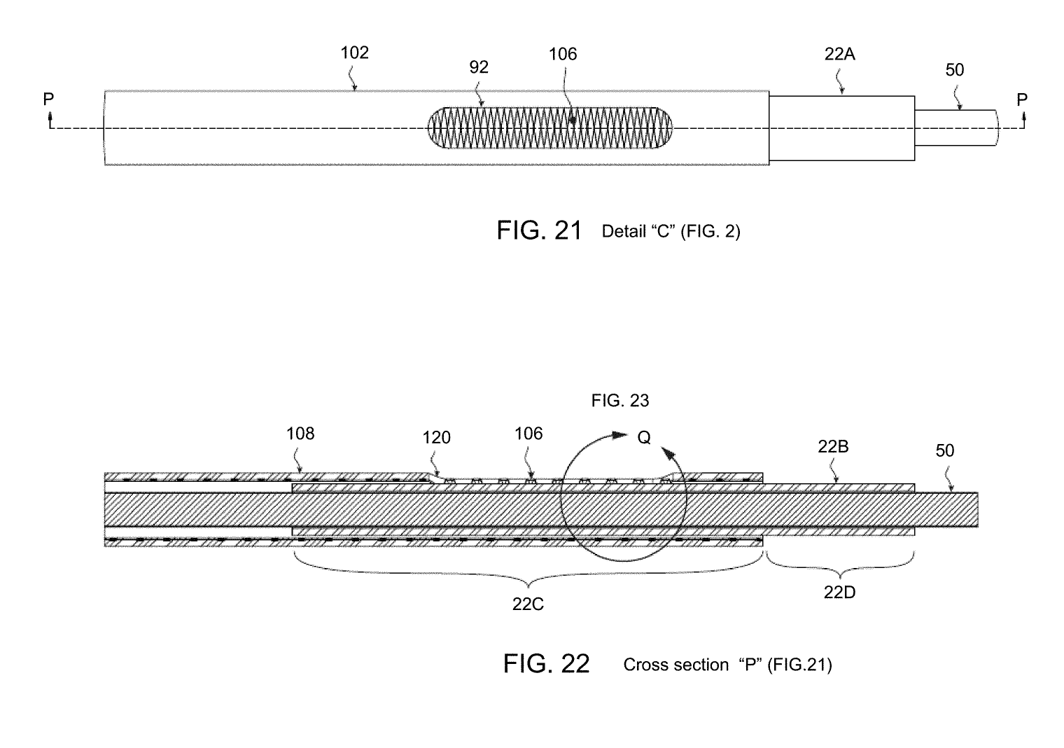

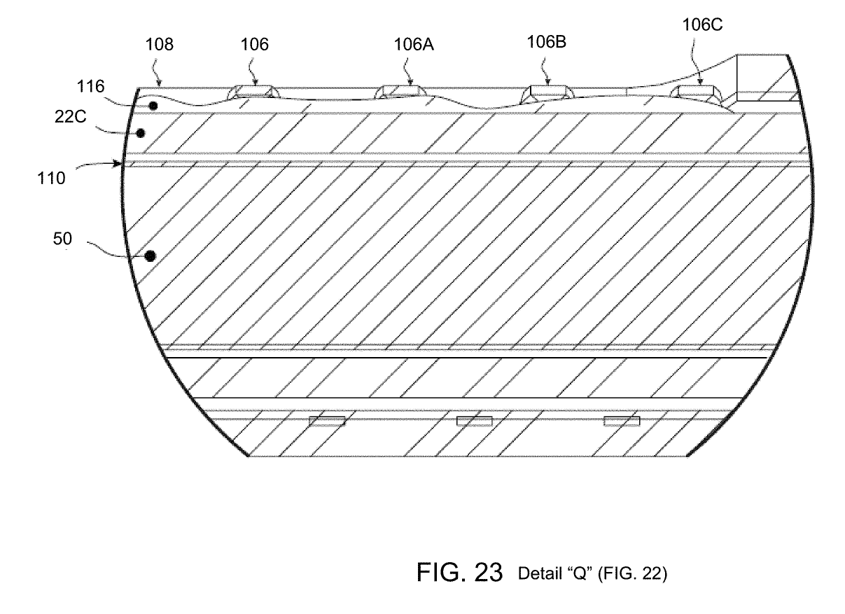

FIG. 22 is a cross sectional view across detail “P” of the proximal end of a guidewire assembly with a solder joint electrical contact embodiment of FIG. 21. A wall 108 of the metallic braid reinforced polymer tubing includes an ablated window section 120. An exposed metallic braid 106 is evident in the ablated window section 120. An electrically conductive first contact region 22B portion of a tube-like structure inserted into a lumen of braid reinforced tubing, extends from the braid reinforced tubing. The tube-like structure is accessible in an area of ablated window that exposes the metallic braid. The metallic tubing inserted within the lumen of the braid reinforced tubing extends into area beneath the ablated window section 22C of the tubing. The electrically conductive contact 22C extends beyond the end of the braid reinforced polymer tubing. The electrically conductive contact 22C is over the insulated core wire 50.

FIG. 23 is detail “Q” in the proximal end of a guidewire with a solder joint electrical contact embodiment of FIG. 22. Core wire 50 of the trackable electromagnetic element is shown having an outer insulation layer 110. A wall of the electrically conductive tubing forms the first electrical contact 22C, which is in electrical connection with the metallic braid via a solder joint 116. That is, solder joint 116 creates an electrical contact between the exposed metallic braid within the wall of the braid reinforced polymer tubing and the tubing of first electrical contact. Particular exposed metallic braids 106, 106A, 1066, 106C of the braid reinforced polymer tubing is shown. A polymer wall 108 of the metallic braid reinforced polymer tubing is shown.

FIG. 24 is detail “D” in the trackable electromagnetic element embodiment 32A of FIG. 2. FIG. 24 illustrates a proximal end of a guidewire assembly at a first electrical contact, which also includes an insulating spacer (contacts) and a non-insulated core wire section. Specifically, a first electrical contact region 22 embodiment in electrical connection with the metallic braid, an insulating spacer 122, and insulation layer 76 of the core wire, and a section 50U where the insulation layer is removed from the core wire at the proximal end of the guidewire assembly is shown.

FIG. 25 is a cross sectional view across detail “R” of the proximal end of a guidewire embodiment of FIG. 24. In FIG. 25, a first electrical contact and insulating spacer arrangement at the proximal end of the guidewire assembly are shown. The embodiment includes an electrically conductive tubing wall 22 that forms the contact, which makes electrical connection with an electrically conductive structure (e.g., metallic braid). A polymer tubing wall forms a contact spacer 122, the core wire has an outer insulation layer 76 that electrically isolates the core wire 50 from other electrically conductive structures, and section of core wire 50U has the insulation layer removed.

FIG. 26 is detail “E” in the trackable electromagnetic element embodiment 32A of FIG. 2. The figure details a contact arrangement at the proximal end of the guidewire assembly. In the assembly, a first electrical contact 22 is formed from an electrically conductive tube-like structure. The first electrical contact 22 is in electrical connection with the metallic braid. An insulating spacer 122 arranged in the polymer tubing separates the first electrical contact region 22 from a second electrical contact region 124. The second electrical contact region 124 is in electrical connection with the core wire 50 of the trackable electromagnetic element. A solder joint 126 is visible, which creates electrical connection with core wire 50. Other techniques to form the electrical connection (e.g., laser welding) are also contemplated.

FIG. 27 is a cross sectional view across detail “S” of the proximal end of a guidewire embodiment of FIG. 26. The figure depicts a detailed view of a wall of the electrically conductive tube-like structure that forms the first electrical contact region 22. The first electrical contact region 22 is in electrical connection with a braid within the polymer reinforced tubing. In the figure, an insulating spacer 122 is formed as integrated with the wall of the polymer tubing. A wall of the electrically conductive tube-like structure forms a second electrical contact region 124, which is in electrical connection with the core wire of the trackable electromagnetic element embodiment. Core wire 50 and a solder joint 126 make electrical connection with the second electrical contact region. An insulating layer 76 of the core wire is shown.

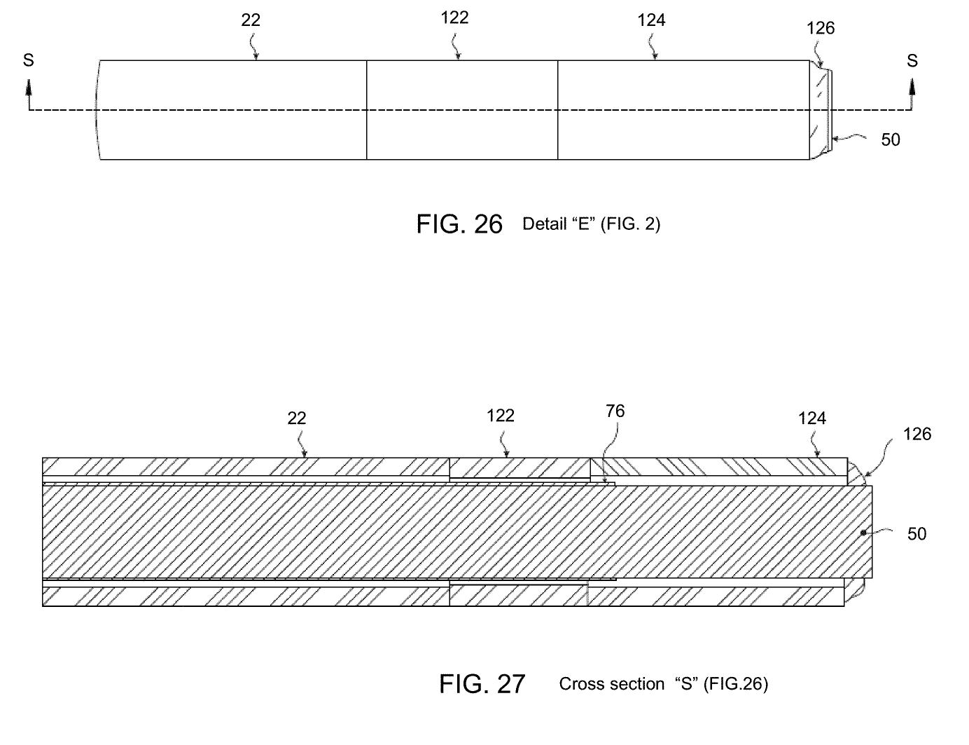

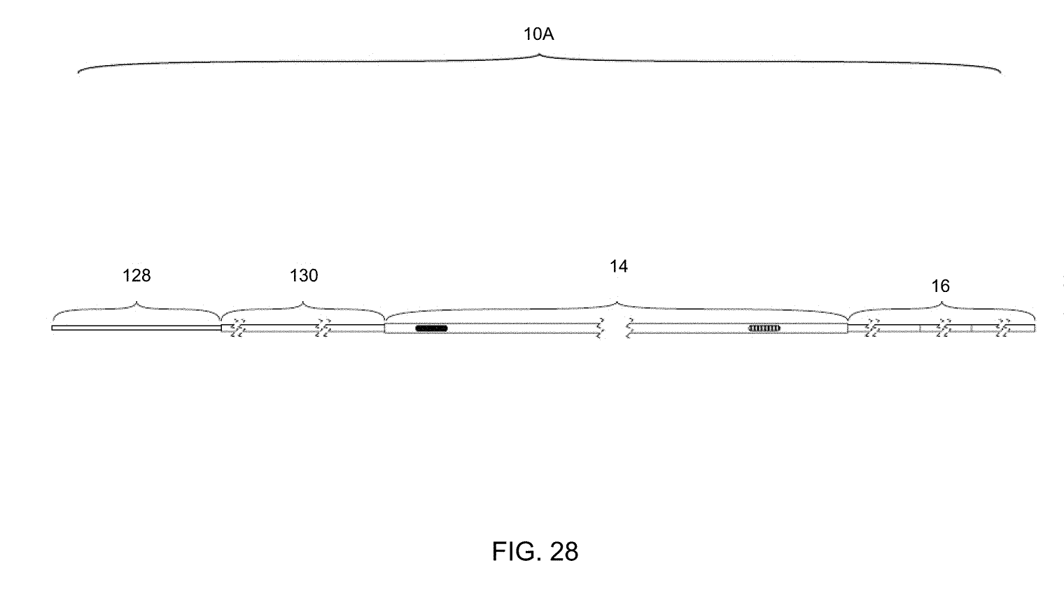

FIG. 28 is a guidewire assembly 10A embodiment used as a core for a medical guidewire. The figure depicts a guidewire core assembly 10A with an extended distal wire. The extended wire section 128 is at the distal tip of the assembly. A coil section 130 of a trackable electromagnetic element embodiment is identified. A metallic braid reinforced polymer tubing 14 section is identified. And an electrical contact arrangement 16 section having the first electrical contact, the insulating spacer, and the second electrical contact is also identified.

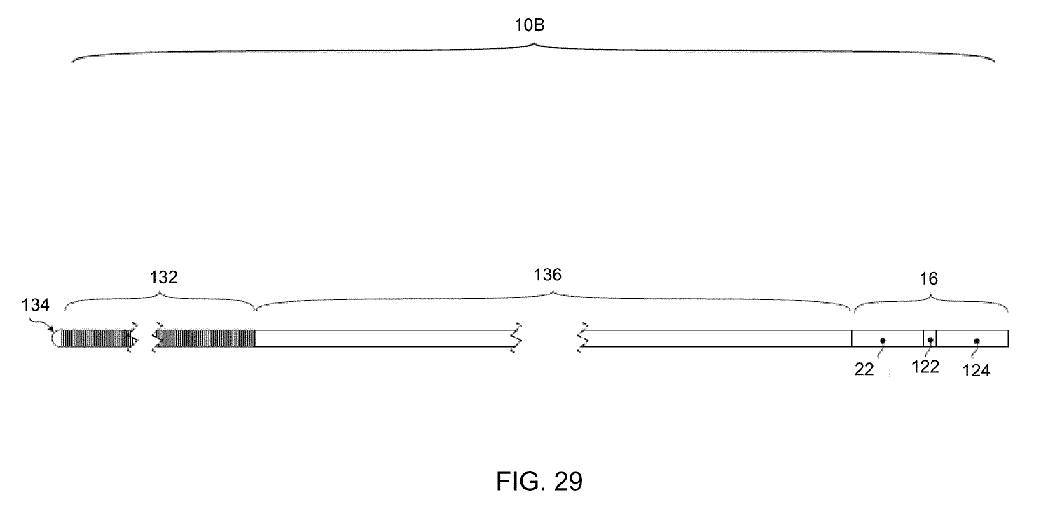

FIG. 29 is a medical guidewire assembly 10B embodiment with a coil pipe overtube. The guidewire assembly 10B includes a coil pipe 132 section at the distal tip of the guide wire assembly, a weld 134 section of the guidewire core assembly, a guidewire body 136 of the polymer tubing overtube, and an electrical contact arrangement 16. The electrical contact arrangement includes a first electrical contact region 22, an insulating spacer 122, and a second electrical contact region 124.

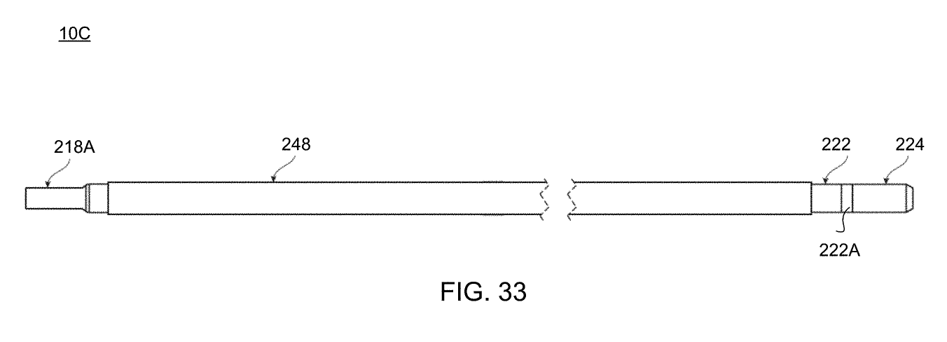

FIG. 30 is a guidewire assembly 10C embodiment having a metallic braid woven over an entire length of a guide wire. The guidewire assembly 10C may be defined between a distal end of the guide wire 218A and a proximal end for the guide wire 218B. FIG. 31 is detail “T” in the guidewire assembly embodiment 10C of FIG. 30; FIG. 32 is detail “U” in the guidewire assembly embodiment 10C of FIG. 30; and FIG. 33 is a guidewire assembly final product embodiment.

The guidewire assembly 10C embodiment of FIGS. 30-33 includes a braided layer that incorporates the inventive principles taught in the present disclosure into a conventional guidewire manufacturing technology. The present embodiment may be distinguished from other embodiments of the present disclosure having an electrically conductive structure formed as a braided metal feature integrated below or in the wall of a polymer tube, which is ablation-exposed and then soldered for electrical connection.

As evident in FIGS. 30-33, a metallic braid is woven over a substantial length of a guide wire. This metallic braid may be formed with a variable weave density over the substantial length of the guide wire. The variable weave is selected for regional, mechanical stiffness variations. For example, in some embodiments, a dense weave in the metallic braid at a distal tip permits improved flexibility at the distal tip. In these or other embodiments, a lower density weave at a proximal end permits a mechanically stiffer structure. Arrangements of such variable weave densities may improve navigation of guidewire assembly 10C through small vessels in a patient's body.

In one method of forming the guidewire assembly 10C of FIGS. 30-33, a coil having an outer insulation layer is wound over a core wire having an outer insulation layer as described in the present disclosure. At least a portion of the coil will form a trackable electromagnetic element. In FIG. 30, the coil portion that forms the trackable electromagnetic element is at section 230 is obscured by the metallic braid 206 and not visible.

In the manufacturing method, the outer layer of insulation of the core wire is removed at the distal portion of the core wire, extending to an area under the distal end of the coil. A portion of the distal end of the wound coil, which may be copper wire in some embodiments, is electrically connected (e.g., soldered, welded, or otherwise electrically joined) to the core wire. Hence the distal end of the coil structure is electrically united to the core wire, and the core wire will be used as a conduit to pass excitation signals through the coil. In FIG. 30,

In FIG. 30, the distal connection of coil-to-core 236 (i.e., coil electrically joined to core) may further be covered by an insulating material such as a polymer sleeve. The insulating material may provide electrical insulation and physical protection for the electrical connection.

In FIG. 30, an outer layer of core wire insulation is also removed at the proximal end of the coil where a second electrical connection is formed. The coil-to-braid connection 238 may be formed by a suitable electrical joint connection (e.g., a soldered connection, a welded connection, or some other electrical joint). Following or otherwise cooperative with the formation of the coil-to-braid connection 238, a covering such as a flexible polymer sleeve or some other covering may be arranged over the coil leaving only a portion of the proximal end of the coil exposed. The covering may be melted or otherwise adhered to seal the coil structure within the covering. Such sealing may provide electrical stability of the trackable electromagnetic element of the guidewire assembly 10C as well as mechanical stability and protection from environmental contaminants.

Subsequent to formation of the trackable electromagnetic element of the guidewire assembly 10C, a braiding operation may be performed. For this, in at least one embodiment, a plurality of strands of metal wire (e.g., 3 strands, 5 strands, 7 strands, or some other number of strands) are woven into a braid or braid-like pattern along a substantial length of the guide wire. In at least one embodiment, the metallic braid 206 is formed substantially between the distal end 218A of the guidewire assembly 10C coil to the proximal end 2186.

In some cases, the metallic braid 206 may have areas of varying wind density. The varying wind density may result in regions of different mechanical stiffness. In at least one embodiment, the metallic braid 206 is formed with a high density at the distal tip to provide increased flexibility, and a low density at the proximal end to provide decreased flexibility.

FIG. 31 is detail “T” in the guidewire assembly embodiment 10C of FIG. 30, and illustrates at least one embodiment of a metallic braid 206 formed with a high density 292. FIG. 32 is detail “U” in the guidewire assembly embodiment 10C of FIG. 30, and illustrates at least one embodiment of a metallic braid 206 formed with a low density 293.

In the proximal region of the coil, which is exposed under the covering (e.g., flexible polymer sleeve) as described herein, an electrical connection is formed electrically join the metallic braid 206 to the proximal end of the guidewire assembly embodiment 10C. This braid-to-electrical connection 216 may form an electrical contact. In at least some embodiments, a conductive covering (e.g., a metallic cylindrical tube) may be slid or otherwise arranged over the core wire and under the metallic braid 206. This conductive cylindrical tube may then be electrically joined to the electrical contact at the braid-to-electrical connection 216. The electrical joint may be formed by soldering, welding, or some other technique.

An insulating (e.g., polymer) “ring” 222A may be slid or otherwise arranged over the core wire at the proximal end of the guidewire assembly embodiment 10C. The ring 222A may be followed by another conductive cylinder 222, which is electrically joined (e.g., soldered, welded, or the like) to the un-insulated section of the core wire. This electrical connection permits excitation signals to be passed between this second conductive cylinder (i.e., now an electrical “contact”) and the core wire, which is electrically coupled to the distal end of the coil at the distal tip of the guidewire assembly embodiment 10C.

The “contact” electrically joined to the metallic braid 206 and electrically joined to the core are electrically isolated by the insulating ring 22A. When an excitation current is driven between the contacts, a magnetic field is induced in the coil, thereby electromagnetically saturating the core, and thereby creating a trackable electromagnetic element at the distal end of the guidewire assembly 10C.

FIG. 33 is a guidewire assembly final product embodiment. In the embodiment, the entire guide wire is encased in a flexible covering 248, such as a polymer tube. The flexible covering 248 may be melted, reflowed, “laminated,” or otherwise arranged to seal the plurality of layers together. This flexible covering 248 can be a formed from variety of materials having a variety of characteristics (e.g., stiffness, softness, hypo-allergenic substances, smoothness, resilience to bodily fluids, resistance to abrasion, and the like). The flexible covering 248 may be coated with a hydro-specific (e.g., hydrophilic/hydrophobic) coating. The flexible covering 248 may have additional features added such as a second braided layer, fluid channel layers, or any other suitable features.

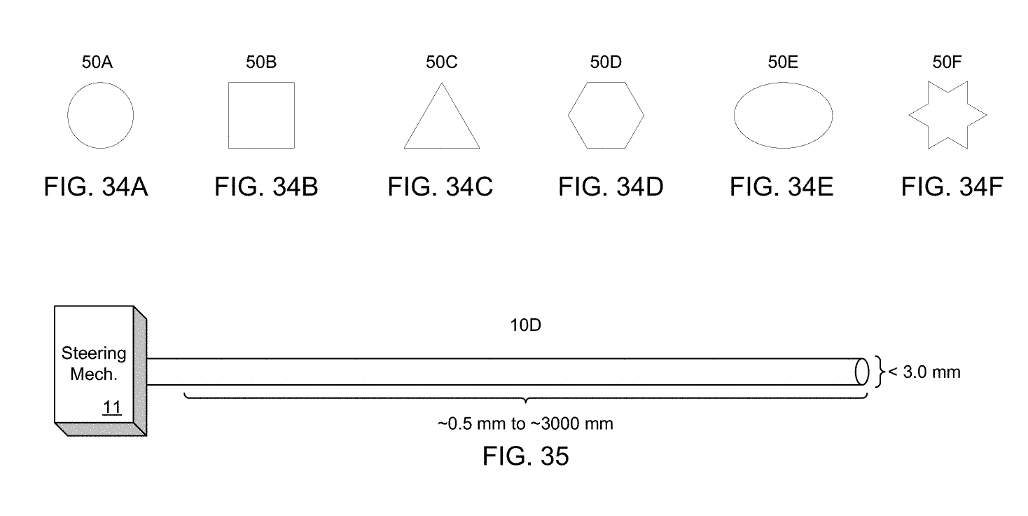

FIGS. 34A-34F are cross sectional views of core wire embodiments 50A-50F. It is recognized that core wire 50 in any of the guidewire assembly embodiments discussed in the present disclosure may take any suitable form. In FIG. 34A, the core wire 50A has a circular cross-section. In FIG. 34B, the core wire 50B has a rectangular (e.g., square) cross-section. In FIG. 34C, the core wire 50C has a triangular cross-section. In FIG. 34D, the core wire 50D has a hexagonal cross-section. In FIG. 34E, the core wire 50E has an ovular cross-section. In FIG. 34F, the core wire 50A has a polygonal cross-section. Other cross-sections are contemplated.

FIG. 35 is another guidewire assembly 10D embodiment. The guidewire assembly of FIG. 35 may be arranged according to any of the system, device, or method embodiments (i.e., the teaching of the present disclosure) described herein. The guidewire assembly 10D may be arranged having any suitable diameter such as, for example, a diameter that is less than about 3.0 millimeters. The guidewire assembly 10D may be arranged having any suitable length such as, for example, a length that is between about 0.5 millimeters and about 3000 millimeters. Other diameters and lengths are also considered.

The guidewire assembly 10D of FIG. 35 is arranged with a steering assembly 11. The steering assembly 11 may be mechanically coupled to the electrically conductive structures (e.g., metallic braid) described herein. Via the mechanical coupling, a medical practitioner can bend, turn, rotate, or perform other actions along the length of the guidewire assembly or additionally or alternatively at the distal end of the guidewire assembly. For example, in some cases, two or more points of contact in the steering assembly 11 may include rotating wheels, sliders, or other means that are respectively coupled to two or more points of separate and distinct lengths of the electrically conductive structures. In this way, a first length may be shortened (e.g., by a rotated wheel or moved slider) while a second length is not. Such movement may cause the distal end of the guidewire assembly 10D to bend in one direction or another. In other cases, a rotation of the steering mechanism 11 may cause torque along the guidewire assembly 10D, which causes a rotation of the guidewire assembly 10D.

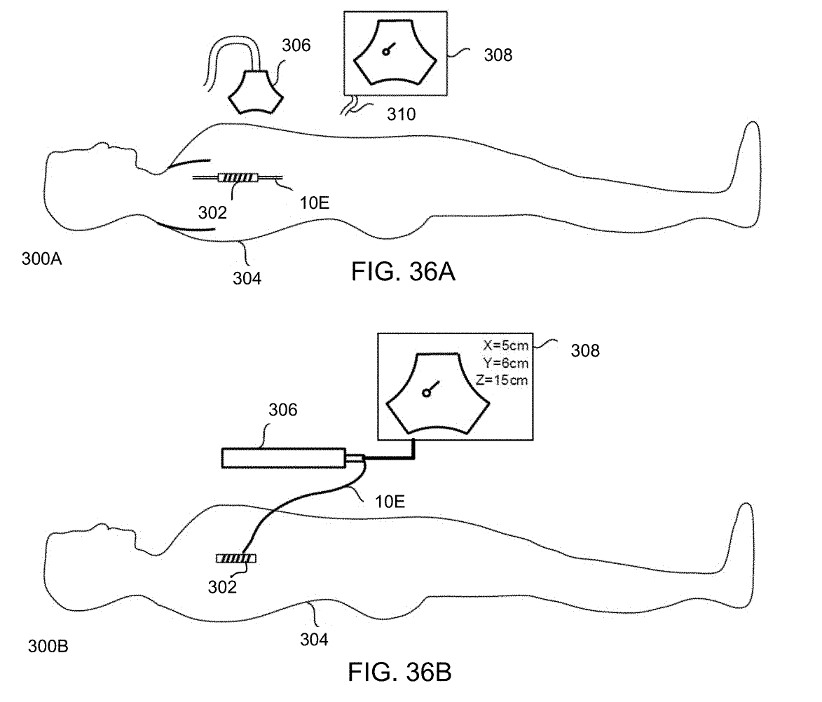

FIG. 36A is a low-frequency electromagnetic tracking system 300A. The low-frequency electromagnetic tracking system 300A includes components for detecting the position of a trackable electromagnetic element 302 formed at the distal end of a guidewire assembly 10E as the guidewire assembly 10E is advanced into the body of a patient. The trackable electromagnetic element 302 and the guidewire assembly 10E are formed according to the teaching of the present disclosure. The patient 304 is undergoing a medical procedure. The patient 304 may be a human patient or a non-human patient.

A medical practitioner (not shown) is administering the procedure. The medical practitioner has advanced the guidewire assembly 10E with the trackable electromagnetic element 302, which may be embodied as a medical instrument, into the body of the patient 304. The guidewire assembly 10E with the trackable electromagnetic element 302 may be used to place a stylet, a catheter such as a Peripherally Inserted Central Catheter (PICC), a medical tube, a tracheal tube, a needle, a cannula, or some other medical device into the body of the patient 304. In some cases, the guidewire assembly 10E with the trackable electromagnetic element 302 includes a lumen or other hollow tube-like structure that can be used to insert a medical device. In some cases, the guidewire assembly 10E with the trackable electromagnetic element 302 is an elongated solid member as described in the present disclosure. In some cases, the guidewire assembly 10E with the trackable electromagnetic element 302 takes another form.

In FIG. 36A, the guidewire assembly 10E with the trackable electromagnetic element 302 may be placed through the mouth of the patient 304 or through another of the patient's orifices. Alternatively, the guidewire assembly 10E with the trackable electromagnetic element 302 may be placed through a surgical incision made by a medical practitioner at some location on the body of the patient 304. The guidewire assembly 10E with the trackable electromagnetic element 302 may be placed and moved in other ways.

A magnetic field sensing device 306 is operated by a medical practitioner proximal to the body of the patient 304. In some cases, the medical practitioner places the magnetic field sensing device 306 directly in contact with the body of the patient 304. Generally speaking, the medical practitioner will attempt to place the magnetic field sensing device 306 adjacent to the portion of the body where the trackable electromagnetic element 302 of the guidewire assembly 10E is believed to be.

A presentation system 308 includes one or more of a video display, an audio input/output system, a tactile feedback system, or some other presentation mechanism. The presentation system 308 may further include one or more user input interfaces for keyboards, mice, touch screens, buttons, dials, and other like controls. The presentation system 308 provides input information to the magnetic field sensing device 306 and receives output information from the magnetic field sensing device 306. Embodiments of the presentation system 308 are used to present information representing the position and orientation of a guidewire assembly 10E with the trackable electromagnetic element 302 by receiving and processing magnetic field information provided by trackable electromagnetic element 302. In at least some cases, the presentation system 308 is arranged with a display to and further arranged to present real time visual imagery representing a path of progress of the distal end of the trackable electromagnetic element 302 of the guidewire assembly 10E advancing in the body of the patient 304.

In some embodiments, the magnetic field sensing device 306 includes an electrical conduit 310. The electrical conduit 310 may be used to pass power signals, control signals, data signals, or some other type of electrical signals. In the embodiment of FIG. 36A, the electrical conduit 310 is arranged to pass electrical signaling information to the trackable electromagnetic element 302 of the guidewire assembly 10E. The electrical conduit 310 may pass electrical signals in a point-to-point arrangement, serial arrangement, parallel arrangement, networked arrangement, and alternatively, in some other arrangement.

The electrical conduit 310 may be used to pass signaling information between the magnetic field sensing device 306 and the presentation system 308. The electrical conduit 310 may in addition or, in the alternative, pass signaling information between the magnetic field sensing device 306 and the trackable electromagnetic element 302 of the guidewire assembly 10E. The signaling information may include power signals, control signals, data signals, or other signals.

In some embodiments, the magnetic field sensing device 306 may include one or more wireless transceivers arranged to communicate data between the magnetic field sensing device 306 and the presentation system 308. In these and other embodiments, the magnetic field sensing device 306 may include one or more wireless transceivers arranged to wirelessly communicate information (e.g., information to generate a particular excitation signal) between the magnetic field sensing device 306 and the trackable electromagnetic element 302 of the guidewire assembly 10E.

FIG. 36B illustrates a medical environment including a low-frequency electromagnetic tracking system 300B for detecting the position of a guidewire assembly 10E with a trackable electromagnetic element 302, which may be arranged to deliver any suitable medical instrument, within the body of a patient 304, according to at least one embodiment. In FIG. 36B, the low-frequency electromagnetic tracking system 300B is a low-frequency electromagnetic tracking system. A patient 304 is positioned on a bed (not shown) and receiving medical treatment.

In receiving the medical treatment, the medical practitioner may identify a particular location of interest inside the body of a patient 304. The guidewire assembly 10E with the trackable electromagnetic element 302 is introduced into and within the body of the patient 304 and advanced. Advancing the distal end of the guidewire assembly 10E into the body of the patient may include stopping the advancing, reversing the direction of progress of the distal end of the guidewire assembly 10E, and restarting the advancing of the distal end of the guidewire assembly 10E. The advancing, stopping, reversing, restarting, and any other such motion may be visually presented in real time visual imagery on the presentation device 308.

A sensor 306 is positioned in proximity to (e.g., above) the patient 304. The sensor 306 includes an wired or wireless communications conduit (not shown) by which the sensor 306 is communicatively coupled to the trackable electromagnetic element 302 of the guidewire assembly 10E and further coupled to a presentation device 308 such as a display. The presentation device 308 may be a mounted display, a tablet device, a smartphone, or some other presentation device and arranged to present real time visual imagery to indicate to the medical practitioner that the trackable electromagnetic element 302 of the guidewire assembly 10E is passing through the patient's body (e.g., through a fluid-carrying conduit such as a blood-carrying conduit (e.g., vein, artery, or the like)) on its way to the identified location. The visual imagery may further confirm to the medical practitioner when the trackable electromagnetic element 302 of the guidewire assembly 10E has reached the identified location.

The sensor 306 includes a control circuit that generates an excitation signal, which is applied to the trackable electromagnetic element 302 of the guidewire assembly 10E. The excitation signal causes a current to flow through the coil of the trackable electromagnetic element 302 of the guidewire assembly 10E. The current causes the trackable electromagnetic element 302 of the guidewire assembly 10E to generate a magnetic field. The magnetic field varies in accordance with the waveform of the excitation signal.

The sensor 306 includes one or more magnetic sensors, which are configured to detect the generated magnetic field and to output one or more corresponding sensor signals to the control circuit. The control circuit analyzes the sensor signals from the one or more magnetic sensors and determines location-based information such as the position, orientation, and motion of the trackable electromagnetic element 302 of the guidewire assembly 10E within the body of the patient 304. The determination of the location-based information is based on the sensor signals and the known characteristics of the excitation signal applied to the electromagnet structure.

In at least one embodiment, the control circuit outputs a video signal to the presentation device 308. The presentation device 308 receives the video signal and displays a representation of the position of the trackable electromagnetic element 302 of the guidewire assembly 10E within the body of the patient 304. The video signal can include position data indicating position coordinates of the trackable electromagnetic element 302 of the guidewire assembly 10E within the body of the patient 304. The presentation device 308 displays the position data so that a medical practitioner, medical personnel, or other technicians can view the position data and the representation of the position of the trackable electromagnetic element 302 of the guidewire assembly 10E in order to appropriately proceed with the medical procedure.

In at least one embodiment, the control circuit can output position data to one or more computing systems (e.g., an ultrasound device, a robotic surgical system) that control or manage aspects of the medical procedure. The one or more computing systems can adjust medical equipment in accordance with the position data. Additionally or alternatively, the computing system can output an alert indicating to medical personnel that there is a potential problem with the position of the trackable electromagnetic element 302 of the guidewire assembly 10E within the body of the patient.

The terms, “real-time” or “real time,” as used herein and in the claims that follow, are not intended to imply instantaneous processing, transmission, reception, or otherwise as the case may be. Instead, the terms, “real-time” and “real time” imply that the activity occurs over an acceptably short period of time (e.g., over a period of microseconds or milliseconds), and that the activity may be performed on an ongoing basis (e.g., presenting visual imagery on a presentation device). An example of an activity that is not real-time is one that occurs over an extended period of time (e.g., hours or days) or that occurs based only on intervention or direction by a medical practitioner or other activity.

In the absence of any specific clarification related to its express use in a particular context, where the terms “substantial” or “about” in any grammatical form are used as modifiers in the present disclosure and any appended claims (e.g., to modify a structure, a dimension, a measurement, or some other characteristic), it is understood that the characteristic may vary by up to 30 percent. For example, a guidewire assembly with a trackable electromagnetic element having a particular linear dimension of “between about 0.5 millimeters and 3000 millimeters” includes such devices in which the linear dimension varies by up to 30 percent. Accordingly, the particular linear dimension of the guidewire assembly with a trackable electromagnetic element may be between 0.35 millimeters and 3900 millimeters.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, the technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

In the present disclosure, when an element (e.g., component, circuit, device, apparatus, structure, layer, material, or the like) is referred to as being “on,” “coupled to,” or “connected to” another element, the elements can be directly on, directly coupled to, or directly connected to each other, or intervening elements may be present. In contrast, when an element is referred to as being “directly on,” “directly coupled to,” or “directly connected to” another element, there are no intervening elements present.

The terms “include” and “comprise” as well as derivatives and variations thereof, in all of their syntactic contexts, are to be construed without limitation in an open, inclusive sense, (e.g., “including, but not limited to”). The term “or,” is inclusive, meaning and/or. The phrases “associated with” and “associated therewith,” as well as derivatives thereof, can be understood as meaning to include, be included within, interconnect with, contain, be contained within, connect to or with, couple to or with, be communicable with, cooperate with, interleave, juxtapose, be proximate to, be bound to or with, have, have a property of, or the like.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the present disclosure, the terms first, second, etc., may be used to describe various elements, however, these elements are not be limited by these terms unless the context clearly requires such limitation. These terms are only used to distinguish one element from another. For example, a first machine could be termed a second machine, and, similarly, a second machine could be termed a first machine, without departing from the scope of the inventive concept.

The singular forms “a,” “an,” and “the” in the present disclosure include plural referents unless the content and context clearly dictates otherwise. The conjunctive terms, “and” and “or” are generally employed in the broadest sense to include “and/or” unless the content and context clearly dictates inclusivity or exclusivity as the case may be. The composition of “and” and “or” when recited herein as “and/or” encompasses an embodiment that includes all of the elements associated thereto and at least one more alternative embodiment that includes fewer than all of the elements associated thereto.

In the present disclosure, conjunctive lists make use of a comma, which may be known as an Oxford comma, a Harvard comma, a serial comma, or another like term. Such lists are intended to connect words, clauses or sentences such that the thing following the comma is also included in the list. The headings and Abstract of the Disclosure provided herein are for convenience only and do not interpret the scope or meaning of the embodiments.

The various embodiments described above can be combined to provide further embodiments. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A medical guidewire assembly, comprising:
an electrically conductive core;
a polymer tubing enclosing at least a portion of the medical guidewire assembly; and
a trackable electromagnetic element formed at a distal end of the electrically conductive core, wherein the trackable electromagnetic element includes a coil, and wherein a first end of the coil is electrically coupled to an electrically conductive structure enclosed by the polymer tubing and a second end of the coil is electrically coupled to the electrically conductive core.

2. The medical guidewire assembly of claim 1, comprising:
at least a first electrical contact region formed at the first end of the coil, wherein the first electrical contact region is formed by a first plurality of coil windings that are shorting together; and
at least a second electrical contact region formed at the second end of the coil, wherein the second electrical contact region is formed by a second plurality of coil windings that are shorting together.

3. The medical guidewire assembly of claim 2, comprising:
at least a third electrical contact region and a fourth electrical contact region formed at the proximal end of the medical guidewire assembly.

4. The medical guidewire assembly of claim 1 wherein the electrically conductive core has a non-circular cross-section.

5. The medical guidewire assembly of claim 1 wherein the electrical coupling of the first end of the coil to the electrically conductive structure is soldered connection.

6. The medical guidewire assembly of claim 1 wherein the electrical coupling of the first end of the coil to the electrically conductive structure is laser weld connection.

7. The medical guidewire assembly of claim 1 wherein the electrically conductive structure and the polymer tubing are integrated into a single structure.

8. The medical guidewire assembly of claim 1 wherein the electrically conductive structure is a metallic braid.

9. The medical guidewire assembly of claim 1 wherein the medical guidewire has a diameter of less than three millimeters.

10. The medical guidewire assembly of claim 1 wherein the medical guidewire has a length between about 0.5 millimeters and about 3000 millimeters.

11. A method to make a medical guidewire assembly bearing a trackable electromagnetic element, comprising:
providing an electrically conductive core, the electrically conductive core having a first outer insulating layer;
providing an electrically conductive guidewire structure;
forming a coil at a distal end of the electrically conductive core, the coil formed from a wire having a second outer insulating layer;
electrically coupling a first end of the coil to the electrically conductive guidewire structure; and
electrically coupling a second end of the coil to the electrically conductive core.

12. The method to make a medical guidewire assembly bearing a trackable electromagnetic element of claim 11, comprising:
enclosing at least a portion of the electrically conductive guidewire with a polymer tubing layer.

13. The method to make a medical guidewire assembly bearing a trackable electromagnetic element of claim 11 wherein electrically coupling the first end of the coil to the electrically conductive guidewire structure comprises:
ablating a portion of the second outer insulating layer of the wire at the first end of the coil to expose an electrically conductive portion of the wire;
exposing an electrically conductive portion of the electrically conductive guidewire; and
forming an electrically conductive joint between the exposed electrically conductive portion of the wire and the exposed electrically conductive portion of the electrically conductive guidewire.

14. The method to make a medical guidewire assembly bearing a trackable electromagnetic element of claim 11 wherein electrically coupling the second end of the coil to the electrically conductive core comprises:
ablating a portion of the second outer insulating layer of the wire at the second end of the coil to expose an electrically conductive portion of the wire;
ablating a portion of the first outer insulating layer of the electrically conductive core to expose an electrically conductive portion of the electrically conductive core; and
forming an electrically conductive joint between the exposed electrically conductive portion of the wire and the exposed electrically conductive portion of the electrically conductive core.

15. The method to make a medical guidewire assembly bearing a trackable electromagnetic element of claim 11 wherein the electrically conductive guidewire structure is a metallic braid.

16. A medical guidewire method, comprising:
identifying a location of interest inside a body of a patient;
introducing a guidewire into the body of the patient, the guidewire having an electrically conductive core, and a trackable electromagnetic element formed in a distal end of the medical guidewire;
advancing the distal end of the medical guidewire into the body of the patient;
magnetically tracking the distal end of the medical guidewire advancing in the body of the patient; and
presenting real time visual imagery representing a path of progress of the distal end of the medical guidewire advancing in the body of the patient.

17. The medical guidewire method of claim 16, comprising:
stopping the advancing when, based on the real time visual imagery, the distal end of the medical guidewire has reached the location of interest inside the body of the patient.

18. The medical guidewire method of claim 16 wherein the advancing includes:
stopping the advancing;
reversing the direction of progress of the distal end of the medical guidewire; and
restarting the advancing of the distal end of the medical guidewire, wherein the stopping, reversing, and restarting are visually presented in the real time visual imagery.

19. The medical guidewire method of claim 16 wherein the advancing includes:
advancing the distal end of the medical guidewire through a fluid-carrying conduit before reaching the location of interest inside the body of the patient.

20. The medical guidewire method of claim 19 wherein the fluid-carrying conduit is a blood-carrying conduit.
